(19) United States
(12) Patent Application Publication
(10) Pub. No.: US 2010/0226928 A1
(43) Pub. Date: Sep. 9, 2010

(54) Lyophilised Antibody Formulation

(75) Inventor: Bhas A. DANI, Basel (CH)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: Novartis AG

(21) Appl. No.: 12/718,387

(22) Filed: Mar. 5, 2010

Related U.S. Application Data
(60) Provisional application No. 61/157,677, filed on Mar. 5, 2009.

(51) Int. Cl. A61K 39/395; C07K 16/18; A61P 19/08

(52) U.S. Cl. 424/152.1; 530/388.2

(57) ABSTRACT

Anti-sclerostin antibodies are formulated as lyophilisates. The lyophilisates can be reconstituted to give a solution with a high concentration of the antibody active ingredient for delivery to a patient without high levels of antibody aggregation. The lyophilisate can be reconstituted with an aqueous reconstituent to provide an aqueous composition in which the antibody has a concentration of at least 25 mg/ml. The lyophilisate may include one or more of a sugar, a buffering agent, a surfactant, and/or a free amino acid.

### RELATED APPLICATIONS

This U.S. nonprovisional application claims priority to U.S. provisional application Ser. No. 61/157,677 filed 5 Mar. 2009, the contents of which are incorporated herein by reference in their entirety.

### TECHNICAL FIELD

This invention is in the field of monoclonal antibody pharmaceutical formulation.

### BACKGROUND

Sclerostin is a key negative regulator of Wnt signalling in bone and is a target for therapeutics designed to treat conditions associated with low bone mass, such as osteoporosis. Monoclonal antibodies which bind to sclerostin are known for use in therapy e.g. see references 1 to 10.

BPS804 is one such antibody (IgG2). It was disclosed as “MOR05813” in reference 10 (the complete contents of which are incorporated herein by reference). It has a V_{H} domain with amino acid SEQ ID NO: 1 and a V_{L} domain with amino acid SEQ ID NO: 2. The variable domains may be expressed as SEQ ID NOs: 9 and 10 to give a functional anti-sclerostin antibody.

Aggregation is a major route of degradation in pharmaceutical formulations of monoclonal antibodies, especially at high concentrations. Aggregation can potentially lead to increased immune response in patients, leading to safety concerns. Thus it must be minimised or prevented.

It is an object of the invention to provide further and improved formulations of anti-sclerostin antibodies, and in particular formulations with low levels of antibody aggregation.

### DISCLOSURE OF THE INVENTION

Monoclonal antibodies (mAbs) are typically formulated either in aqueous form ready for parenteral administration or as lyophilisates for reconstitution with a suitable diluent prior to administration. According to the invention, an anti-sclerostin antibody is formulated as a lyophilisate. Suitable formulation can provide a lyophilisate which can be reconstituted to give a solution with a high concentration of the antibody active ingredient for delivery to a patient without high levels of antibody aggregation. High concentrations of antibody are useful as they reduce the amount of material which must be delivered to a patient. Reduced dosing volumes minimise the time taken to deliver a fixed dose to the patient.

Thus the invention provides a lyophilisate comprising an anti-sclerostin monoclonal antibody, wherein the lyophilisate can be reconstituted with an aqueous reconstituent to provide an aqueous composition in which the antibody has a concentration of at least 25 mg/ml.

The invention also provides an aqueous pharmaceutical composition comprising an anti-sclerostin monoclonal antibody, wherein the antibody has a concentration of at least 25 mg/ml.

The invention also provides a lyophilisate comprising: an anti-sclerostin monoclonal antibody; a sugar; a buffering agent; and a surfactant. The lyophilisate preferably also includes a free amino acid.

The invention also provides an aqueous pharmaceutical composition comprising: an anti-sclerostin monoclonal antibody; a sugar; a buffering agent; and a surfactant. The composition preferably also includes a free amino acid.

The invention also provides a lyophilisate comprising an anti-sclerostin monoclonal antibody, wherein the lyophilisate can be reconstituted with an aqueous reconstituent to provide an aqueous composition in which less than 1% of the anti-sclerostin monoclonal antibody is aggregated.

The invention also provides an aqueous pharmaceutical composition comprising an anti-sclerostin monoclonal antibody, wherein less than 1% of the anti-sclerostin monoclonal antibody is aggregated.

The invention also provides a process for preparing a lyophilisate, comprising steps of: (i) preparing an aqueous solution comprising an anti-sclerostin monoclonal antibody, a sugar, a buffering agent, a surfactant, and optionally a free amino acid; and (ii) lyophilising the aqueous solution.

The invention also provides a process for preparing a composition, comprising a step of mixing a lyophilisate with an aqueous reconstituent, wherein the lyophilisate comprises an anti-sclerostin monoclonal antibody, a sugar, a buffering agent, a surfactant, and optionally a free amino acid.

### Lyophilisates

Techniques for lyophilisation of mAbs are well known in the art e.g. see references 11 to 19. For example, monoclonal antibody products SYNAGIS™, REMICADE™, RAPTIVA™, SIMULECT™, XOLAIR™ and HERCEPTIN™ are supplied as lyophilisates. These antibodies are reconstituted to various final concentrations e.g. SIMULECT™ is reconstituted to a concentration of 4 mg/ml antibody, REMICADE™ is reconstituted to a concentration of 10 mg/ml, HERCEPTIN™ to 21 mg/ml, SYNAGIS™ and RAPTIVA™ to 100 mg/ml, and XOLAIR™ to 125 mg/ml.

Lyophilisates of the invention can be reconstituted to give aqueous compositions with an anti-sclerostin antibody concentration of at least 25 mg/ml. The antibody concentration can be much higher than 25 mg/ml e.g. ≧50 mg/ml, ≧75 mg/ml, ≧100 mg/ml, ≧125 mg/ml, ≧150 mg/ml or higher.

Furthermore, the lyophilisates of the invention are stable such that even after storage for 4 weeks at 2-8° C., they can be reconstituted to give aqueous compositions in which less than 1% of the total anti-sclerostin antibody is aggregated (as measured by SEC-HPLC) e.g. <0.5%, <0.4%, <0.3%, etc.

The lyophilisate may include, in addition to the anti-sclerostin mAb, further components such as one or more of the following: (i) a sugar; (u) a buffering agent; (iii) a surfactant; and (iv) a free amino acid. Inclusion of each of such additional components (i), (ii) and (iii) is typical, and can give compositions with low aggregation of the anti-sclerostin mAb. Inclusion of component (iv) is advantageous because it has been shown to further reduce aggregation after storage.

Suitable sugars for use with the invention include, but are not limited to, monosaccharides, disaccharides and trisaccharides. For example, the sugar may be sucrose, trehalose, raffinose, maltose, sorbitol or mannitol. The sugar may be a sugar alcohol or an amino sugar. Sucrose is particularly useful.

Suitable buffering agents for use with the invention include, but are not limited to, a histidine buffer, a citrate buffer, a phosphate buffer, a succinate buffer, an acetate buffer, or a Tris buffer. A histidine buffer is particularly useful.

Suitable surfactants for use with the invention include, but are not limited to, non-ionic surfactants, ionic surfactants and zwitterionic surfactants. Typical surfactants for use with the invention include, but are not limited to, sorbitan fatty acid esters (e.g. sorbitan monocaprylate, sorbitan monolaurate, sorbitan monopalmitate), sorbitan trioleate, glycerine fatty acid esters (e.g. glycerine monocaprylate, glycerine monomyristate, glycerine monostearate), polyglycerine fatty acid esters (e.g. decaglyceryl monostearate, decaglyceryl distearate, decaglyceryl monolinoleate), polyoxyethylene sorbitan fatty acid esters (e.g. polyoxyethylene sorbitan monolaurate, polyoxyethylene sorbitan monooleate, polyoxyethylene sorbitan monostearate, polyoxyethylene sorbitan monopalmitate, polyoxyethylene sorbitan trioleate, polyoxyethylene sorbitan tristearate), polyoxyethylene sorbitol fatty acid esters (e.g. polyoxyethylene sorbitol tetrastearate, polyoxyethylene sorbitol tetraoleate), polyoxyethylene glycerine fatty acid esters (e.g. polyoxyethylene glyceryl monostearate), polyethylene glycol fatty acid esters (e.g. polyethylene glycol distearate), polyoxyethylene alkyl ethers (e.g. polyoxyethylene lauryl ether), polyoxyethylene polyoxypropylene alkyl ethers (e.g. polyoxyethylene polyoxypropylene glycol, polyoxyethylene polyoxypropylene propyl ether, polyoxyethylene polyoxypropylene cetyl ether), polyoxyethylene alkylphenyl ethers (e.g. polyoxyethylene nonylphenyl ether), polyoxyethylene hydrogenated castor oils (e.g. polyoxyethylene castor oil, polyoxyethylene hydrogenated castor oil), polyoxyethylene beeswax derivatives (e.g. polyoxyethylene sorbitol beeswax), polyoxyethylene lanolin derivatives (e.g. polyoxyethylene lanolin), and polyoxyethylene fatty acid amides (e.g. polyoxyethylene stearic acid amide); C₁₀-C₁₈ alkyl sulfates (e.g. sodium cetyl sulfate, sodium lauryl sulfate, sodium oleyl sulfate), polyoxyethylene C₁₀-C₁₈ alkyl ether sulfate with an average of 2 to 4 moles of ethylene oxide units added (e.g. sodium polyoxyethylene lauryl sulfate), and C₁-C₁₈ alkyl sulfosuccinate ester salts (e.g. sodium lauryl sulfosuccinate ester); and natural surfactants such as lecithin, glycerophospholipid, sphingophospholipids (e.g. sphingomyelin), and sucrose esters of C₁₂-C₁₈ fatty acids. A composition may include one or more of these surfactants. Preferred surfactants are polyoxyethylene sorbitan fatty acid esters e.g. polysorbate 20, 40, 60 or 80. Polysorbate 80 (Tween 80) is particularly useful.

Suitable free amino acids for use with the invention include, but are not limited to, arginine, lysine, histidine, ornithine, isoleucine, leucine, alanine, glycine glutamic acid or aspartic acid. The inclusion of a basic amino acid is preferred i.e. arginine, lysine and/or histidine. If a composition includes histidine then this may act both as a buffering agent and a free amino acid, but when a histidine buffer is used it is typical to include a non-histidine free amino acid e.g. to include histidine buffer and lysine. An amino acid may be present in its D- and/or L-form, but the L-form is typical. The amino acid may be present as any suitable salt e.g. a hydrochloride salt, such as Arginine-HCl.

When present, components (i) to (iv) will be at a pre-lyophilisation concentration sufficient to maintain the anti-sclerostin antibody in a form which is active and soluble after storage (under normal conditions) and reconstitution. The components will also be present after reconstitution.

Thus a sugar may be present before lyophilisation at a concentration of between 3 and 300 mM e.g. 15-200 mM, 30-150 mM, 80-100 mM. A concentration of 90 mM sucrose or trehalose is useful.

A buffering agent may be present before lyophilisation at a concentration of between 1 and 60 mM e.g. 3-30 mM, 6-20 mM, 8-15 mM. A concentration of 10 mM histidine buffer is useful.

A surfactant may be present before lyophilisation at a concentration of up to 0.2% (by volume) e.g. 0.01-0.1%, 0.01-0.08%, 0.01-0.04%. A concentration of 0.02% polysorbate 80 is useful.

A free amino acid may be present before lyophilisation at a concentration of between 2 and 80 mM e.g. 3-50 mM, 6-30 mM, 10-25 mM, 15-20 mM. A concentration of 17 mM arginine-HCl is useful.

A formulation containing histidine buffer, sucrose and polysorbate 80 has been shown to be suitable for lyophilisation of antibody BPS804. Additional inclusion of arginine reduces BPS804 aggregation.

A lyophilisate may include active ingredients in addition to the mAb. For instance, further pharmacological agents may be included, such as chemotherapeutic compounds. For instance, methotrexate may be included, and it is known to include methotrexate sodium in lyophilisates.

The pH of an aqueous mAb formulation prior to lyophilisation may be in the range 4.0-8.0, which a pH in the range 6.0-7.4 being typical. Some anti-sclerostin antibodies are not stable in aqueous solution above pH 6.0 and so a composition may have a pH in the range of 5.0 to 6.0. For instance, a pre-lyophilisation pH of 5.3±0.1 is suitable for BPS804.

### Aqueous Reconstitution

Before a lyophilisate can be administered to a patient it should be reconstituted with an aqueous reconstituent. This step permits antibody and other components in the lyophilisate to re-dissolve to give a solution which is suitable for injection to a patient.

The volume of aqueous material used for reconstitution dictates the concentration of mAb in a resulting pharmaceutical composition. Reconstitution with a smaller volume of reconstituent than the pre-lyophilisation volume provides a composition which is more concentrated than before lyophilisation. As mentioned above, lyophilisates of the invention can be reconstituted to give aqueous compositions with an anti-sclerostin antibody concentration of at least 25 mg/ml (or higher), and the volume of reconstituent will be selected accordingly.

Typical reconstituents for lyophilised mAbs include sterile water or buffer, optionally containing a preservative. If the lyophilisate includes a buffering agent then the reconstituent may include further buffering agent (which may be the same as or different from the lyophilisate's buffering agent) or it may instead include no buffering agent (e.g. WFI, physiological saline).

When present, components (i) to (iv) mentioned above will be at a concentration sufficient to maintain the anti-sclerostin antibody in active soluble form, after reconstitution, under normal storage conditions while retaining pharmaceutical acceptability at the point of use.

Thus a sugar may be present after reconstitution at a concentration of between 10 and 800 mM e.g. 50-500 mM, 100-400 mM, 200-300 mM. A concentration of 270 mM sucrose or trehalose is useful.

A buffering agent may be present after reconstitution at a concentration of between 2 and 200 mM e.g. 5-150 mM, 10-100 mM, 15-50 mM, 20-40 mM, 25-35 mM. A concentration of 30 mM histidine buffer is useful.

A surfactant may be present after reconstitution at a concentration of up to 0.5% (by volume) e.g. 0.001-0.2%, 0.01-0.1%, 0.04-0.08%, 0.05-0.07%. A concentration of 0.06% polysorbate 80 is useful.

A free amino acid may be present after reconstitution at a concentration of between 5 and 250 mM e.g. 10-150 mM, 20-100 mM, 40-80 mM, 50-70 mM. A concentration of 51 mM arginine-HCl is useful.

An aqueous reconstituent may include pharmacological agents, such as chemotherapeutic compounds, facilitating co-delivery together with the mAb.

After reconstitution, compositions of the invention include anti-sclerostin antibody, and less than 1% of the total anti-sclerostin antibody is aggregated (as measured by SEC-HPLC) e.g. <0.5%, <0.4%, <0.3%, etc.

Ideally, aqueous reconstitution does not result in formation of a gel. If the pH of the aqueous reconstituent for BPS804 is too high, for instance, the reconstituted material can spontaneously form a gel, but it is preferred that aqueous compositions of the invention will remain as liquid solutions.

### Pharmaceutical Compositions

Lyophilisates of the invention can be reconstituted to give aqueous pharmaceutical compositions. Such compositions are pharmaceutically acceptable and are suitable for administration to a patient. In addition to mAb and water they may include further components, derived from the lyophilisate and/or the reconstituent. Such components include, but are not limited to, buffers, salts, amino acids, glycerol, alcohols, preservatives, surfactants, etc. A thorough discussion of such pharmaceutical ingredients is available in reference 20.

The use of mAbs as the active ingredient of pharmaceuticals is now widespread, including the products HERCEPTIN™ (trastuzumab), RITUXAN™ (rituximab), SYNAGIS™ (palivizumab), etc. Techniques for purification of mAbs to a pharmaceutical grade are well known in the art.

The composition will usually be sterile, at least at the time of its formation. The composition will usually be non-pyrogenic e.g. containing <1 EU (endotoxin unit, a standard measure) per dose, and preferably <0.1 EU per dose. The composition is preferably gluten-free.

Within formulations of the invention, a mAb preferably makes up at least 80% by weight (e.g. at least 90%, 95%, 97%, 98%, 99% or more) of the total protein in the formulation. The mAb is thus in purified form.

### Target Diseases and Disorders

Anti-sclerostin antibodies can be used to treat or prevent a variety of diseases or disorders. These include diseases and disorders in which bone mineral density (BMD) is abnormally and/or pathologically high relative to healthy subjects, such as sclerosteosis, Van Buchem disease, bone overgrowth disorders, and Simpson-Golabi-Behmel syndrome (SGBS). They also include diseases and disorders in which bone mineral density (BMD) is abnormally and/or pathologically low relative to healthy subjects, such as osteoporosis (primary and/or secondary), osteopenia, osteomalacia, osteogenesis imperfecta (OI), avascular necrosis (osteonecrosis), fractures and implant healing (dental implants and hip implants), bone loss due to other disorders (e.g. associated with HIV infection, cancers, or arthritis). Further sclerostin-related disorders include, but are not limited to, rheumatoid arthritis, osteoarthritis, arthritis, hypophosphatasia (including adult-onset hypophosphatasia) and the formation and/or presence of osteolytic lesions.

The disease/disorder will generally be mediated by sclerostin, or be associated with or characterized by aberrant sclerostin levels. These include cancers and osteoporotic conditions (e.g. osteoporosis or osteopenia). Sclerostin-related cancers can include myeloma (e.g. multiple myeloma with osteolytic lesions), breast cancer, colon cancer, melanoma, hepatocellular cancer, epithelial cancer, esophageal cancer, brain cancer, lung cancer, prostate cancer, or pancreatic cancer, as well as any metastases thereof.

A sclerostin-related disorder can also include renal and cardiovascular conditions, due at least to sclerostin's expression in the kidney and cardiovasculature. Said disorders include, but are not limited to, such renal disorders as glomerular diseases (e.g. acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes (e.g. type 2 diabetes), polycystic kidney disease, neoplasia, sickle cell disease, and chronic inflammatory diseases), tubular diseases (e.g. acute tubular necrosis and acute renal failure, polycystic renal disease, medullary sponge kidney, medullary cystic disease, nephrogenic diabetes, and renal tubular acidosis), tubulointerstitial diseases (e.g. pyelonephritis, drug and toxin induced tubulointerstitial nephritis, hypercalcemic nephropathy, and hypokalemic nephropathy) acute and rapidly progressive renal failure, chronic renal failure, nephrolithiasis, gout, vascular diseases (e.g. hypertension and nephrosclerosis, microangiopathic hemolytic anemia, atheroembolic renal disease, diffuse cortical necrosis, and renal infarcts), or tumors (e.g. renal cell carcinoma and nephroblastoma).

Target diseases/disorders also include cardiovascular disorders such as ischemic heart disease (e.g. angina pectoris, myocardial infarction, and chronic ischemic heart disease), hypertensive heart disease, pulmonary heart disease, valvular heart disease (e.g. rheumatic fever and rheumatic heart disease, endocarditis, mitral valve prolapse, and aortic valve stenosis), congenital heart disease (e.g. valvular and vascular obstructive lesions, atrial or ventricular septal defect, and patent ductus arteriosus), or myocardial disease (e.g. myocarditis, congestive cardiomyopathy, and hypertrophic cardiomyopathy).

### Patient Administration

A pharmaceutical composition of the invention can be administered to a patient. Administration will typically be via a syringe. Thus the invention provides a delivery device (e.g. a syringe) including a pharmaceutical composition of the invention.

Patients will receive an effective amount of the mAb active ingredient i.e. an amount that is sufficient to detect, treat, ameliorate, or prevent the disease or disorder in question. Therapeutic effects may also include reduction in physical symptoms. The optimum effective amount and concentration of mAb for any particular subject will depend upon various factors, including the patient's age size health and/or gender, the nature and extent of the condition, the activity of the particular mAb, the rate of its clearance by the body, and also on any possible further therapeutic(s) administered in combination with the mAb. The effective amount delivered for a given situation can be determined by routine experimentation and is within the judgment of a clinician. For purposes of the present invention, an effective dose may be from about 0.01 mg/kg to about 50 mg/kg, or about 0.05 mg/kg to about 10 mg/kg. Known antibody-based pharmaceuticals provide guidance in this respect e.g. HERCEPTIN™ is administered with an initial loading dose of 4 mg/kg body weight and a weekly maintenance dose of 2 mg/kg body weight; RITUXAN™ is administered weekly at 375 mg/m²; SYNAGIS™ is administered intramuscularly at 15 mg/kg body weight; etc.

The invention provides a method for delivering a monoclonal antibody to a mammal, comprising a step of administering to the patient a pharmaceutical composition of the invention.

The invention also provides a method for delivering a monoclonal antibody to a mammal, comprising steps of: (i) reconstituting a lyophilisate of the invention to give an aqueous formulation, and (ii) administering the aqueous formulation to the patient. Step (ii) ideally takes place within 24 hours of step (i) e.g. within 12 hours, within 6 hours, within 3 hours, or within 1 hour.

The invention also provides formulations of the invention for use as medicaments e.g. for use in delivering a monoclonal antibody to a mammal, or for use in treating one or more of the diseases and disorders described above.

The mammal is preferably a human but may also be, for example, a horse or a cow or a dog or a cat. The mAb will ideally be chosen to match the target species e.g. a human antibody for human administration, an equine antibody for horses, a canine antibody for dogs, etc. If native host antibodies are not available then transfer of antibody specificity from one species to another can be achieved by transfer of CDR residues (and typically, in addition, one or more framework residues) from a donor antibody into a recipient framework from the host species e.g. as in humanisation. Equinised, bovinised, caninised and felinised antibodies are known in the art. The antibody will bind to sclerostin from the target species, but it may also cross-react with sclerostin from other species.

Dosage can be by a single dose schedule or a multiple dose schedule.

Ingredients for forming compositions of the invention (e.g. lyophilisates and reconstituents) may be supplied in hermetically-sealed containers.

### The Monoclonal Antibody

The invention concerns the formulation of anti-sclerostin monoclonal antibodies. The term “monoclonal” as originally used in relation to antibodies referred to antibodies produced by a single clonal line of immune cells, as opposed to “polyclonal” antibodies that, while all recognizing the same target protein, were produced by different B cells and would be directed to different epitopes on that protein. As used herein, the word “monoclonal” does not imply any particular cellular origin, but refers to any population of antibodies that display a single binding specificity and affinity for a particular epitope in the same target protein. This usage is normal e.g. the product datasheets for the CDR-grafted humanised antibody SYNAGIS™ expressed in a murine myeloma NSO cell line, for the humanised antibody HERCEPTIN™ expressed in a CHO cell line, and for the phage-displayed antibody HUMIRA™ expressed in a CHO cell line, all refer to the active ingredients as “monoclonal” antibodies.

Thus a mAb may be produced using any suitable protein synthesis system, including immune cells, non-immune cells, acellular systems, etc. A mAb can thus be produced by a variety of techniques, including conventional monoclonal antibody methodology (e.g. the standard somatic cell hybridization technique of Kohler & Milstein), by viral or oncogenic transformation of B lymphocytes, by combinatorial synthesis, by phage display, etc.

Antibodies used with the invention can take various forms. For instance, they may be native antibodies, as naturally found in mammals. Native antibodies are made up of heavy chains and light chains. The heavy and light chains are both divided into variable domains and constant domains. The ability of different antibodies to recognize different antigens arises from differences in their variable domains, in both the light and heavy chains. Light chains of native antibodies in vertebrate species are either kappa (κ) or lambda (λ), based on the amino acid sequences of their constant domains. The constant domain of a native antibody's heavy chains will be α, δ, ε, γ or μ, giving rise respectively to antibodies of IgA, IgD, IgE, IgG, or IgM class. Classes may be further divided into subclasses or isotypes e.g. IgG1, IgG2, IgG3, IgG4, IgA, IgA2, etc. Antibodies may also be classified by allotype e.g. a γ heavy chain may have G1m allotype a, f, x or z, G2m allotype n, or G3m allotype b0, b1, b3, b4, b5, c3, c5, g1, g5, s, t, u, or v; a κ light chain may have a Km(1), Km(2) or Km(3) allotype. A native IgG antibody has two identical light chains (one constant domain C_{L} and one variable domain V_{L}) and two identical heavy chains (three constant domains C_{H}1 C_{H}2 & C_{H}3 and one variable domain V_{H}), held together by disulfide bridges. The domain and three-dimensional structures of the different classes of native antibodies are well known.

Where an antibody of the invention has a light chain with a constant domain, it may be a κ or λ light chain. Where an antibody of the invention has a heavy chain with a constant domain, it may be an α, δ, ε, γ or μ heavy chain. Heavy chains in the γ class (i.e. IgG antibodies) are preferred.

Antibodies of the invention may be fragments of native antibodies that retain antigen binding activity. For instance, papain digestion of native antibodies produces two identical antigen-binding fragments, called “Fab” fragments, each with a single antigen-binding site, and a residual “Fc” fragment without antigen-binding activity. Pepsin treatment yields a “F(ab′)₂” fragment that has two antigen-binding sites. “Fv” is the minimum fragment of a native antibody that contains a complete antigen-binding site, consisting of a dimer of one heavy chain and one light chain variable domain. Thus an antibody of the invention may be Fab, Fab′, F(ab′)₂, Fv, or any other type, of fragment of a native antibody.

An antibody of the invention may be a “single-chain Fv” (“scFv” or “sFv”), comprising a V_{H} and V_{L} domain as a single polypeptide chain [21-23]. Typically the V_{H} and V_{L} domains are joined by a short polypeptide linker (e.g. ≧12 amino acids) between the V_{H} and V_{L} domains that enables the scFv to form the desired structure for antigen binding. A typical way of expressing scFv proteins, at least for initial selection, is in the context of a phage display library or other combinatorial library [24-26]. Multiple scFvs can be linked in a single polypeptide chain [27].

An antibody of the invention may be a “diabody” or “triabody” etc. [28-31], comprising multiple linked Fv (scFv) fragments. By using a linker between the V_{H} and V_{L} domains that is too short to allow them to pair with each other (e.g. <12 amino acids), they are forced instead to pair with the complementary domains of another Fv fragment and thus create two antigen-binding sites. These antibodies may include C_{H} and/or C_{L} domains.

An antibody of the invention may be a single variable domain or VHH antibody. Antibodies naturally found in camelids (e.g. camels and llamas) and in sharks contain a heavy chain but no light chain. Thus antigen recognition is determined by a single variable domain, unlike a mammalian native antibody [32-34]. The constant domain of such antibodies can be omitted while retaining antigen-binding activity. One way of expressing single variable domain antibodies, at least for initial selection, is in the context of a phage display library or other combinatorial library [35].

An antibody of the invention may be a “domain antibody” (dAb). Such dAbs are based on the variable domains of either a heavy or light chain of a human antibody and have a molecular weight of approximately 13 kDa (less than one-tenth the size of a full antibody). By pairing heavy and light chain dAbs that recognize different targets, antibodies with dual specificity can be made. dAbs are cleared from the body quickly and so benefit from a sustained release system, but can additionally be sustained in circulation by fusion to a second dAb that binds to a blood protein (e.g. to serum albumin), by conjugation to polymers (e.g. to a polyethylene glycol), or by other techniques.

The antibody may have a scaffold which is based on the fibronectin type III domain, as disclosed in reference 36 e.g. an adnectin or trinectin. The fibronectin-based scaffold is not an immunoglobulin, although the overall fold is closely related to that of the smallest functional antibody fragment. Because of this structure the non-immunoglobulin antibody mimics antigen binding properties that are similar in nature and affinity to those of natural antibodies. The FnIII domain has 7 or 8 beta strands which are distributed between two beta sheets, which themselves pack against each other to form the core of the protein, and further containing loops (analogous to antibody CDRs) which connect the beta strands to each other and are solvent exposed. There are at least three such loops at each edge of the beta sheet sandwich, where the edge is the boundary of the protein perpendicular to the direction of the beta strands. The FnIII loops can be replaced with immunoglobulin CDRs using standard cloning techniques, and can be used in a loop randomization and shuffling strategy in vitro that is similar to the process of affinity maturation of antibodies in vivo. The FnIII scaffold may be based on the tenth module of fibronectin type III (i.e. 10Fn3).

Thus the term “antibody” as used herein encompasses a range of proteins having diverse structural features, but usually including at least one immunoglobulin domain, having an all-β protein fold with a 2-layer sandwich of anti-parallel β-strands arranged in two β-sheets.

Antibodies used with the invention may include a single antigen-binding site (e.g. as in a Fab fragment or a scFv) or multiple antigen-binding sites (e.g. as in a F(ab′)₂ fragment or a diabody or a native antibody). Where an antibody has more than one antigen-binding site then advantageously it can result in cross-linking of antigens.

Where an antibody has more than one antigen-binding site, the antibody may be mono-specific (i.e. all antigen-binding sites recognize the same antigen) or it may be multi-specific (i.e. the antigen-binding sites recognise more than one antigen).

An antibody of the invention may include a non-protein substance e.g. via covalent conjugation. For example, an antibody may include a radio-isotope e.g. the ZEVALIN™ and BEXXAR™ products include ⁹⁰Y and ¹³¹I isotopes, respectively. As a further example, an antibody may include a cytotoxic molecule e.g. MYLOTARG™ is linked to N-acetyl-γ-calicheamicin, a bacterial toxin. As a further example, an antibody may include a covalently-attached polymer e.g. attachment of polyoxyethylated polyols or polyethylene glycol (PEG) has been reported to increase the circulating half-life of antibodies.

In some embodiments, an antibody can include one or more constant domains (e.g. including C_{H} or C_{L} domains). As mentioned above, the constant domains may form a κ or λ light chain or an α, δ, ε, γ or μ heavy chain. Where an antibody includes a constant domain, it may be a native constant domain or a modified constant domain. A heavy chain may include either three (as in α, γ, δ classes) or four (as in μ, ε classes) constant domains. Constant domains are not involved directly in the binding interaction between an antibody and an antigen, but they can provide various effector functions, including but not limited to: participation of the antibody in antibody-dependent cellular cytotoxicity (ADCC); C1q binding; complement dependent cytotoxicity; Fc receptor binding; phagocytosis; and down-regulation of cell surface receptors.

The constant domains can form a “Fc region”, which is the C-terminal region of a native antibody's heavy chain. Where an antibody of the invention includes a Fc region, it may be a native Fc region or a modified Fc region. A Fc region is important for some antibodies' functions e.g. the activity of HERCEPTIN™ is Fc-dependent. Although the boundaries of the Fc region of a native antibody may vary, the human IgG heavy chain Fc region is usually defined to stretch from an amino acid residue at position Cys226 or Pro230 to the heavy chain's C-terminus. The Fc region will typically be able to bind one or more Fc receptors, such as a FcγRI (CD64), a FcγRII (e.g. FcγRIIA, FcγRIIB1, FcγRIIB2, FcγRIIC), a FcγRIII (e.g. FcγRIIIA, FcγRIIIB), a FcRn, FcαR (CD89), FcδR, FcμR, a FcεRI (e.g. FccRIαβγ₂ or FcεRIαγ₂), FcεRII (e.g. FcεRIIA or FcεRIIB), etc. The Fc region may also or alternatively be able to bind to a complement protein, such as C1q. Modifications to an antibody's Fc region can be used to change its effector function(s) e.g. to increase or decrease receptor binding affinity. For instance, reference 37 reports that effector functions may be modified by mutating Fc region residues 234, 235, 236, 237, 297, 318, 320 and/or 322. Similarly, reference 38 reports that effector functions of a human IgG1 can be improved by mutating Fc region residues (EU Index Kabat numbering) 238, 239, 248, 249, 252, 254, 255, 256, 258, 265, 267, 268, 269, 270, 272, 276, 278, 280, 283, 285, 286, 289, 290, 292, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 and/or 439. Modification of Fc residues 322, 329 and/or 331 is reported in reference 39 for modifying C1q affinity of human IgG antibodies, and residues 270, 322, 326, 327, 329, 331, 333 and/or 334 are selected for modification in reference 40. Mapping of residues important for human IgG binding to FcRI, FcRII, FcRIII, and FcRn receptors is reported in reference 41, together with the design of variants with improved FcR-binding properties. Whole C_{H} domains can be substituted between isotypes e.g. reference 42 discloses antibodies in which the C_{H}3 domain (and optionally the C_{H}2 domain) of human IgG4 is substituted by the C_{H}3 domain of human IgG1 to provide suppressed aggregate formation. Reference 42 also reports that mutation of arginine at position 409 (EU index Kabat) of human IgG4 to e.g. lysine shows suppressed aggregate formation. Mutation of the Fc region of available monoclonal antibodies to vary their effector functions is known e.g. reference 43 reports mutation studies for RITUXAN™ to change C1q-binding, and reference 44 reports mutation studies for NUMAX™ to change FcR-binding, with mutation of residues 252, 254 and 256 giving a 10-fold increase in FcRn-binding without affecting antigen-binding.

Antibodies will typically be glycosylated. N-linked glycans attached to the C_{H}2 domain of a heavy chain, for instance, can influence C1q and FcR binding [41], with a glycosylated antibodies having lower affinity for these receptors. The glycan structure can also affect activity e.g. differences in complement-mediated cell death may be seen depending on the number of galactose sugars (0, 1 or 2) at the terminus of a glycan's biantennary chain. An antibody's glycans preferably do not lead to a human immunogenic response after administration.

Antibodies can be prepared in a form free from products with which they would naturally be associated. Contaminant components of an antibody's natural environment include materials such as enzymes, hormones, or other host cell proteins.

Useful antibodies have nanomolar or picomolar affinity constants for their target antigens e.g. 10⁻⁹ M, 10⁻¹⁰ M, 10⁻¹¹ M, 10⁻¹² M, 10⁻¹³ M or tighter). Such affinities can be determined using conventional analytical techniques e.g. using surface plasmon resonance techniques as embodied in BIAcore™ instrumentation and operated according to the manufacturer's instructions. Radio-immunoassay using radiolabeled target antigen (sclerostin) is another method by which binding affinity may be measured.

The monoclonal antibody used with the invention may be a human antibody, a humanized antibody, a chimeric antibody or (particularly for veterinary purposes) a non-human antibody.

In some embodiments the antibodies are human mAbs. These can be prepared by various means. For example, human B cells producing an antigen of interest can be immortalized e.g. by infection with Epstein Ban Virus (EBV), optionally in the presence of a polyclonal B cell activator [45 & 46]. Human monoclonal antibodies can also be produced in non-human hosts by replacing the host's own immune system with a functioning human immune system e.g. into Scid mice or Trimera mice. Transgenic and transchromosomic mice have been successfully used for generating human monoclonal antibodies, including the “humab mouse” from Medarex and the “xeno-mouse” from Abgenix [47], collectively referred to herein as “human Ig mice”. Phage display has also been successful [48], and led to the HUMIRA™ product. Unlike non-human antibodies, human antibodies will not elicit an immune response directed against their constant domains when administered to humans. Furthermore, the variable domains of these human antibodies are fully human (in particular the framework regions of the variable domains are fully human, in addition to the complementarity determining regions [CDRs]) and so will not elicit an immune response directed against the variable domain framework regions when administered to humans (except, potentially, for any anti-idiotypic response). Human antibodies do not include any sequences that do not have a human origin.

In some embodiments the antibodies are humanised mAbs, CDR-grafted mAbs or chimeric mAbs. These can be prepared by various means. For example, they may be prepared based on the sequence of a non-human (e.g. murine) monoclonal antibody. DNA encoding the non-human heavy and light chain immunoglobulins can be obtained and engineered to contain human immunoglobulin sequences using standard molecular biology techniques. For example, to create a chimeric antibody, the murine variable regions can be linked to human constant regions using methods known in the art. To create a CDR-grafted antibody, the murine CDR regions can be inserted into a human framework [49-54]. To create a humanized antibody, one or more non-CDR variable framework residue(s) is also altered. The H1, H2 and H3 CDRs may be transferred together into an acceptor V_{H} domain, but it may also be adequate to transfer only one or two of them [52]. Similarly, one two or all three of the L1, L2 and L3 CDRs may be transferred into an acceptor V_{L} domain. Preferred antibodies will have 1, 2, 3, 4, 5 or all 6 of the donor CDRs. Where only one CDR is transferred, it will typically not be the L2 CDR, which is usually the shortest of the six. Typically the donor CDRs will all be from the same human antibody, but it is also possible to mix them e.g. to transfer the light chain CDRs from a first antibody and the heavy chain CDRs from a second antibody.

Anti-sclerostin antibodies useful with the present invention may include one or more (1, 2, 3, 4, 5 or 6) CDRs from BPS804. The CDRs in the heavy chain are SEQ ID NOs: 3, 4 & 5. The CDRs in the light chain are SEQ ID NOs: 6, 7 & 8.

In some embodiments the antibodies are non-human mAbs. These can be prepared by various means e.g. the original Kohler & Milstein technique for preparing murine mAbs.

In some embodiments of the invention, the antibody has a variable domain with an isoelectric point (pI) in the range of 5.0 to 8.0. In some embodiments the antibody is an IgG2.

One suitable anti-sclerostin antibody for use with the invention is BPS804. Thus the anti-sclerostin antibody may have a V_{H} domain with amino acid SEQ ID NO: 1 and/or a V_{L} domain with amino acid SEQ ID NO: 2. The antibody may comprise SEQ ID NOs: 9 and 10.

### Further Antibodies

Although the invention is presented above in relation to anti-sclerostin antibodies, the formulation disclosed herein is also suitable for use with antibodies which recognise antigens other than sclerostin. Thus the invention provides a lyophilisate comprising a monoclonal antibody, sucrose, a histidine buffer, polysorbate 80 and arginine. This lyophilisate can be reconstituted with an aqueous reconstituent, and the invention also provides an aqueous pharmaceutical composition comprising a monoclonal antibody, sucrose, a histidine buffer, polysorbate 80 and arginine.

The reconstituted composition obtainable from the lyophilisate may have an antibody concentration of at least 25 mg/ml (as described above) in which less than 1% of the anti-sclerostin is aggregated (as described above).

The invention also provides a process for preparing a lyophilisate, comprising steps of: (i) preparing an aqueous solution comprising a monoclonal antibody, sucrose, a histidine buffer, polysorbate 80 and free arginine; and (ii) lyophilising the aqueous solution. The invention also provides a process for preparing a composition, comprising a step of mixing a lyophilisate with an aqueous reconstituent, wherein the lyophilisate comprises a monoclonal antibody, sucrose, a histidine buffer, polysorbate 80 and free arginine.

Sucrose may be present at a concentration of between 10 and 800 mM e.g. 50-500 mM, 100-400 mM, 200-300 mM. A concentration of 270 mM sucrose is useful.

The histidine buffer may be present at a concentration of between 5 and 50 mM e.g. 10-45 mM, 20 40 mM, 25-35 mM. A concentration of 30 mM histidine buffer is useful.

The polysorbate 80 may be present at a concentration of up to 0.5% (by volume) e.g. 0.01-0.1%, 0.04-0.08%, 0.05-0.07%. A concentration of 0.06% polysorbate 80 is useful.

The arginine may be present at a concentration of between 5 and 250 mM e.g. 10-150 mM, 20 100 mM, 40-80 mM, 50-70 mM. A concentration of 51 mM arginine-HCl is useful.

The pH of an aqueous mAb formulation prior to lyophilisation may be in the range 5.0-8.0.

Typical reconstituents for the lyophilised mAb include sterile water or buffer, as described above.

### General

The practice of the present invention will employ, unless otherwise indicated, conventional methods of chemistry, biochemistry, molecular biology, immunology, pharmacy, posology and pharmacology, within the skill of the art. Such techniques are explained fully in the literature. See, e.g. references 55-61, etc.

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

The word “substantially” does not exclude “completely” e.g. a composition which is “substantially free” from Y may be completely free from Y. Where necessary, the word “substantially” may be omitted from a definition of the invention.

### BRIEF DESCRIPTION OF DRAWINGS

There are no drawings.

### MODES FOR CARRYING OUT THE INVENTION

Antibody ‘BPS804’ recognises sclerostin and is disclosed as ‘MOR05813’ in reference 10. It is a human IgG2λ mAb obtained via phage display. Its heavy and light chains are SEQ ID NOs: 9 and 10.

A high concentration lyophilised formulation of BPS804 was desired and so formulation studies were performed. A lyophilised formulation comprising a sugar, a buffering agent and a surfactant was stable at 2-8° C. and could maintain high antibody concentrations after reconstitution. Addition of arginine-HCl to the formulation reduced aggregation.

Three formulations (F1, F2, F3) of BPS804 at 100 mg/vial were evaluated for stability in a first study. Each formulation had, prior to lyophilisation, 33.3 mg/ml BPS804, pH 5.3, and a fill volume of 3.6 ml. The three formulations included buffer, sugar, surfactant and free amino acid as follows:

| | Buffer | Sugar | Surfactant | Amino acid |
|---|---|---|---|---|
| F1 | 10 mM histidine | 90 mM sucrose | 0.02% polysorbate 80 | — |
| F2 | 10 mM histidine | 90 mM trehalose | 0.02% polysorbate 80 | — |
| F3 | 10 mM histidine | 90 mM sucrose | 0.02% polysorbate 80 | 17 mM arginine-HCl |

The lyophilisates were reconstituted with WFI to giving a reconstituted volume of 1.2 ml (20% overage; ⅓ the original aqueous volume). Thus the reconstituted compositions were as follows, all containing 100 mg/ml antibody:

| | Buffer | Sugar | Surfactant | Amino acid |
|---|---|---|---|---|
| F1 | 30 mM histidine | 270 mM sucrose | 0.06% polysorbate 80 | — |
| F2 | 30 mM histidine | 270 mM trehalose | 0.06% polysorbate 80 | — |
| F3 | 30 mM histidine | 270 mM sucrose | 0.06% polysorbate 80 | 51 mM arginine-HCl |

The three reconstituted formulations were tested for stability (i) prior to lyophilisation, (ii) after immediate post-lyophilisation reconstitution, and (iii) after reconstitution following storage at 2-8° C. or 40° C. for four weeks. Stability was evaluated by % aggregates (measured by SEC-HPLC) and by clarity (assessed by visual inspection after overnight storage at 2-8° C.).

Aggregation results from SEC-HLPC were as follows:

| | | | 2-8° C. | 40° C. |
|---|---|---|---|---|
| | Pre-lyo | Post-lyo | for 4 weeks | for 4 weeks |
| F1 | 0.23% | 0.29% | 0.27% | 0.74% |
| F2 | 0.22% | 0.27% | 0.30% | 1.05% |
| F3 | 0.22% | 0.21% | 0.21% | 0.53% |

Visual clarity was as follows:

| | | | 2-8° C. | 40° C. |
|---|---|---|---|---|
| | Pre-lyo | Post-lyo | for 4 weeks | for 4 weeks |
| F1 | Opalescent | Opalescent | Opalescent | Opalescent |
| F2 | Milky | Milky | Opalescent/Milky | Opalescent/Milky |
| F3 | Clear | Clear | Clear | Clear |

Thus F3 showed the lowest aggregation of BPS804 after reconstitution, measured both by SEC-HPLC and by visual appearance.

Based on these results a second study was performed with a higher antibody concentration using formulations F1 and F3. The pre-lyophilisation antibody concentration was increased to 50 mg/ml but other components were as before. Reconstitution with WFI to a 1.2 ml volume was again used. Thus the reconstituted compositions were as follows, all containing 150 mg/ml antibody:

| | Buffer | Sugar | Surfactant | Amino acid |
|---|---|---|---|---|
| F1′ | 30 mM histidine | 270 mM sucrose | 0.06% polysorbate 80 | — |
| F3′ | 30 mM histidine | 270 mM sucrose | 0.06% polysorbate 80 | 51 mM arginine-HCl |

The same stability tests were performed and results were as follows:

| | | | 2-8° C. | 40° C. |
|---|---|---|---|---|
| | Pre-lyo | Post-lyo | for 4 weeks | for 4 weeks |
| F1′ | 0.25% | 0.34% | 0.34% | 1.57% |
| | Clear | Turbid | Turbid | Slightly turbid |
| F3′ | 0.22% | 0.29% | 0.30% | 1.17% |
| | Clear | Clear | Clear | Clear |

Again, therefore, the F3 formulation gave the best stability.

It will be understood that the invention will be described by way of example only, and that modifications may be made whilst remaining within the scope and spirit of the invention.

### REFERENCES

[1] WO2008/092894.
[2] WO2005/014650.
[3] WO2006/119062.
[4] WO2006/119107.
[5] WO2008/061013.
[6] WO2008/115732
[7] WO2008/133722.
[8] WO2006/102070.
[9] Li et al. (2008) J Bone Miner Res 2008:10.1359/jbmr.081206
[10] WO2009/047356.
[11] Rey & May (2004) Freeze-Drying/Lyophilization Of Pharmaceutical & Biological Products ISBN 0824748689.
[12] WO92/15331.
[13] LIS patent application 2008/0286280.
[14] WO03/041637.
[15] WO2008/116103.
[16] WO2008/029908.
[17] WO2007/074880.
[18] WO03/009817.
[19] WO98/022136.
[20] Gennaro (2000) Remington: The Science and Practice of Pharmacy. 20th edition, ISBN: 0683306472.
[21] Worn & Pluckthun (2001) J Mol Biol. 305(5):989-1010.
[22] WO93/16185
[23] Adams & Schier (1999) J Immunol Methods. 231(1-2):249-60.
[24] Hallborn & Carlsson (2002) Biotechniques Supp1:30-7.
[25] Pini & Bracci (2000) Curr Protein Pept Sci 1(2):155-69.
[26] Walter et al. (2001) Comb Chem High Throughput Screen. 4(2):193-205.
[27] Gruber et al. (1994) J Immunol 152(11):5368-74.
[28] U.S. Pat. No. 5,591,828
[29] WO 93/11161.
[30] Hollinger et al. (1993) Proc. Natl. Acad. Sci. USA 90:6444-6448.
[31] Hudson & Kortt (1999) J Immunol Methods 231:177-89.
[32] Muyldermans (2001) J Biotechnol 74(4):277-302.
[33] Dumoulin et al. (2002) Protein Sci. 11(3):500-15.
[34] Sidhu et al. (2004) J Mol. Biol. 338(2):299-310.
[35] Kotz et al. (2004) Eur J Biochem. 271(9):1623-9.
[36] U.S. Pat. No. 6,818,418.
[37] U.S. Pat. No. 5,624,821.
[38] U.S. Pat. No. 6,737,056.
[39] U.S. Pat. No. 6,538,124.
[40] U.S. Pat. No. 6,528,624.
[41] Shields et al. (2001) J Biol Chem 276:6591-604.
[42] WO2006/033386.
[43] Idusogie et al. (2000) J Immunol 164(8):4178-84.
[44] Dall'acqua et al. (2006) J Biol Chem 281(33):23514-24.
[45] WO2004/076677.
[46] Traggiai et al. (2004) Nat Med 10(8):871-5.
[47] Green (1999) J Immunol Methods. 231(1-2):11-23.
[48] Mancini et al. (2004) New Microbiol. 27(4):315-28.
[49] Ewert et al. (2004) Methods 34(2):184-99.
[50] Riechmann et al. (1988) Nature 332:323-327.
[51] O'Brien & Jones (2003) Methods Mol. Biol. 207:81-100.
[52] Iwahashi et al. (1999) Mol Immunol. 36(15-16):1079-91.
[53] Lo (2004) Methods Mol Biol. 248:135-59.
[54] Verhoeyen et al. (1988) Science 239: 1534-1536.
[55] Methods In Enzymology (S. Colowick and N. Kaplan, eds., Academic Press, Inc.)
[56] Handbook of Experimental Immunology, Vols. I-IV (D. M. Weir and C. C. Blackwell, eds, 1986, Blackwell Scientific Publications)
[57] Sambrook et al. (2001) Molecular Cloning: A Laboratory Manual, 3rd edition (Cold Spring Harbor Laboratory Press).
[58] Handbook of Surface and Colloidal Chemistry (Birdi, K. S. ed., CRC Press, 1997)
[59] Ausubel et al. (eds) (2002) Short protocols in molecular biology, 5th edition (Current Protocols).
[60] Molecular Biology Techniques: An Intensive Laboratory Course, (Ream et al., eds., 1998, Academic Press)
[61] PCR (Introduction to Biotechniques Series), 2nd ed. (Newton & Graham eds., 1997, Springer Verlag)

### Sequence Listing

SEQ ID NO: 1

QVQLVESGGGLVQPGGSLRLSCAASGFTFRSHWLSWVRQAPGKGLEWVSN
INYDGSSTYYADSVKGRFTISRDNSKNTLYLQMNSLFAEDTAVYYCARDT
YLHFDYWGQGTLVTVSS

SEQ ID NO: 2

DIALTQPASVSGSPGQSITISCTGTSSDVGDINDVSWYQQHPGKAPKLMI
YDVNNRPSGVSNRFSGSKSGNTASLTISGLQAEDEADYYCQSYAGSYLSE
VFGGGTKLTVLGQ

SEQ ID NO: 3

GFTFRSHWLS

SEQ ID NO: 4

WVSNINYDGSSTYYADSVKG

SEQ ID NO: 5

DTYLHFDY

SEQ ID NO: 6

TGTSSDVGDINDVS

SEQ ID NO: 7

LMIYDVNNRPS

SEQ ID NO: 8

QSYAGSYLSE

SEQ ID NO: 9

MAWVWTLPFLMAAAQSVQAQVQLVESGGGLVQPGGSLRLSCAASGFTFRS
HWLSWVRQAPGKGLEWVSNINYDGSSTYYADSVKGRFTISRDNSKNTLYL
QMHSLRAEDTAVYYCARDTYLHFDYWGQGTLVTVSSASTKGPSVFPLAPC
SRSTSESTAALGCLVKDYFRFPVTVSWNSGALTSGVHTFPAVLQSSGLYS
LSSVVTVRSSNFGTQTYTCNVDHKPSNTKVDKTVERKCCVECPPCPAPPV
AGPSVFLFPPKPKDTLMISRTPEVTCVVVDVSHEDPEVQFNWYVDGVEVH
NAKTKPREEQFNSTFRVVSVLTVVHQDWLNGKEYKCKVSNKGLPAPIEKT
ISKTKGQPREPQWTLPPSREEMTKNQVSLTCLVKGFYPSDIAVEWESNGQ
PENNYKTTPPMLDSDGSFFLYSKLTVDKSRWQQGNVFSCSVMHEALHNHY
TQKSLSLSPGK

SEQ ID NO: 10

MSVLTQVLALLLLWLTGTRCDIALTQPASVSGSPGQSITISCTGTSSDVG
DINDVSWYQQHPGKAPKLMIYDVNNRPSGVSNRFSGSKSGNTASLTISGL
QAEDEADYYCQSYAGSYLSEVFGGGTKLTVLGQPKAAPSVTLFPPSSEEL
QANKATLVCLISDFYPGAVTVAWKADSSPVKAGVETTTPSKQSNNKYAAS
SYLSLTPEQWKSHRSYSCQVTHEGSTVEKTVAPTECS

1. A lyophilisate comprising an anti-sclerostin monoclonal antibody, wherein the lyophilisate can be reconstituted with an aqueous reconstituent to provide an aqueous composition in which the antibody has a concentration of at least 25 mg/ml.

2. A lyophilisate comprising an anti-sclerostin monoclonal antibody, wherein the lyophilisate can be reconstituted with an aqueous reconstituent to provide an aqueous composition in which less than 1% of the anti-sclerostin antibody is aggregated.

3. A lyophilisate comprising: an anti-sclerostin monoclonal antibody; a sugar; a buffering agent; and a surfactant.

4. The lyophilisate of claim 2, further comprising a free amino acid.

5. The lyophilisate of claim 1, wherein the lyophilisate can be reconstituted with an aqueous reconstituent to provide an aqueous composition in which less than 1% of the anti-sclerostin antibody is aggregated.

6. The lyophilisate of claim 2, wherein the lyophilisate can be reconstituted with an aqueous reconstituent to provide an aqueous composition in which the antibody has a concentration of at least 25 mg/ml.

7. The lyophilisate of claim 2, comprising one, two or three of: a sugar; a buffering agent; and a surfactant.

8. The lyophilisate of claim 7, further comprising a free amino acid.

9. The lyophilisate of claim 1, wherein the lyophilisates can be reconstituted to give an aqueous composition with an anti-sclerostin antibody concentration of at least 125 mg/ml.

10. The lyophilisate of claim 1, comprising sucrose.

11. The lyophilisate of claim 10, comprising 200-300 mM sucrose.

12. The lyophilisate of claim 1, comprising a histidine buffer.

13. The lyophilisate of claim 12, comprising 25-35 mM histidine buffer.

14. The lyophilisate of claim 1, comprising polysorbate 80.

15. The lyophilisate of claim 14, comprising 0.01 to 0.1% polysorbate 80.

16. The lyophilisate of claim 1, comprising arginine.

17. The lyophilisate of claim 15, comprising 40-80 mM arginine.

18. The lyophilisate of claim 1, comprising sucrose, a histidine buffer, polysorbate 80 and arginine.

19. The lyophilisate of claim 1, wherein the anti-sclerostin antibody includes: (i) one or more heavy chain CDRs selected from the group consisting of SEQ ID NOs: 3, 4 & 5; and/or (ii) one or more light chain CDRs selected from the group consisting of SEQ ID NOs: 6, 7 & 8.

20. The lyophilisate of claim 1, wherein the anti-sclerostin antibody has a V_{H} domain with amino acid SEQ ID NO: 1 and/or a V_{L} domain with amino acid SEQ ID NO: 2.

21. An aqueous pharmaceutical composition obtainable by reconstitution of the lyophilisate of claim 1 with an aqueous reconstituent.

22. A process for preparing the lyophilisate of claim 1, wherein the process comprises the steps of: (i) preparing an aqueous solution comprising an anti-sclerostin monoclonal antibody, a sugar, a buffering agent, a surfactant, and, optionally, a free amino acid; and (ii) lyophilising the aqueous solution.

23. A process for preparing a pharmaceutical composition, comprising a step of: mixing the lyophilisate of claim 1 with an aqueous reconstituent.

24. A delivery device including the pharmaceutical composition of claim 21.

25. A method for delivering an anti-sclerostin monoclonal antibody to a mammal, comprising a step of administering to the patient a pharmaceutical composition of claim 21.

26. The composition of claim 21 for use in treating a disease or disorders in which bone mineral density is abnormally and/or pathologically high relative to healthy subjects.

27. The composition of claim 21 for use in treating a disease or disorders in which bone mineral density is abnormally and/or pathologically low relative to healthy subjects.

28. The composition of claim 21 for use in treating osteoporosis.


SEQUENCE LISTING (10 sequences)

SEQ ID NO: 1 - protein, 117 aa, Artificial
  antibody VH
QVQLVESGGGLVQPGGSLRLSCAASGFTFRSHWLSWVRQAPGKGLEWVSNINYDGSSTYYADSVKGRFTISRDNSKNTLYLQMNSLRAEDTAVYYCARDTYLHFDYWGQGTLVTVSS

SEQ ID NO: 2 - protein, 113 aa, Artificial
  antibody VL
DIALTQPASVSGSPGQSITISCTGTSSDVGDINDVSWYQQHPGKAPKLMIYDVNNRPSGVSNRFSGSKSGNTASLTISGLQAEDEADYYCQSYAGSYLSEVFGGGTKLTVLGQ

SEQ ID NO: 3 - protein, 10 aa, Artificial
  CDR
GFTFRSHWLS

SEQ ID NO: 4 - protein, 20 aa, Artificial
  CDR
WVSNINYDGSSTYYADSVKG

SEQ ID NO: 5 - protein, 8 aa, Artificial
  CDR
DTYLHFDY

SEQ ID NO: 6 - protein, 14 aa, Artificial
  CDR
TGTSSDVGDINDVS

SEQ ID NO: 7 - protein, 11 aa, Artificial
  CDR
LMIYDVNNRPS

SEQ ID NO: 8 - protein, 10 aa, Artificial
  CDR
QSYAGSYLSE

SEQ ID NO: 9 - protein, 462 aa, Artificial
  antibody heavy chain
MAWVWTLPFLMAAAQSVQAQVQLVESGGGLVQPGGSLRLSCAASGFTFRSHWLSWVRQAPGKGLEWVSNINYDGSSTYYADSVKGRFTISRDNSKNTLYLQMNSLRAEDTAVYYCARDTYLHFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 237 aa, Artificial
  antibody light chain
MSVLTQVLALLLLWLTGTRCDIALTQPASVSGSPGQSITISCTGTSSDVGDINDVSWYQQHPGKAPKLMIYDVNNRPSGVSNRFSGSKSGNTASLTISGLQAEDEADYYCQSYAGSYLSEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS